(19) United States
(12) Patent Application Publication
(10) Pub. No.: US 2025/0000647 A1
(43) Pub. Date: Jan. 2, 2025

(54) GUIDE CATHETER FOR PROSTHETIC CARDIAC VALVE DELIVERY SYSTEMS

(71) Applicant: Shifamed Holdings, LLC, Campbell, CA (US)

(72) Inventors: Ryan William BOYD, Santa Cruz, CA (US); Nicholas J. SPINELLI, San Carlos, CA (US); Noah GOLDSMITH, Santa Cruz, CA (US); Andrew BACKUS, Santa Cruz, CA (US); Keke LEPULU, Menlo Park, CA (US)

(21) Appl. No.: 18/688,735

(22) PCT Filed: Sep. 1, 2022
(86) PCT No.: PCT/US2022/075865

§ 371 Date: Mar. 1, 2024

Related U.S. Application Data
(60) Provisional application No. 63/260,821, filed on Sep. 1, 2021.

(51) Int. Cl. A61F 2/24

(52) U.S. Cl. CPC A61F 2/2427

(57) ABSTRACT

Systems and methods usable in delivering an anchoring or docking device to a native valve of a patient's heart. A distal region of a delivery catheter can be positioned in an atrium of the heart and a. distal tip can be positioned at or near a commissure of the native valve using a steerable sheath. The one or more bends can be generated and/or adjusted in a distal portion of the steerable sheath to manipulate the position of the distal tip of the delivery catheter.

### CROSS-REFERENCE TO RELATED APPLICATIONS

This application claims priority to U.S. Provisional Application No. 63/260,821, filed Sep. 1, 2021, entitled “GUIDE CATHETER FOR PROSTHETIC CARDIAC VALVE DELIVERY SYSTEMS,” which is incorporated herein by reference in its entirety.

### FIELD

The present disclosure generally concerns deployment tools for delivering anchoring devices, for example anchoring devices that support valve prostheses and methods of using the same. For example, the disclosure relates to replacement of heart valves that have malformations and/or dysfunctions, where a flexible delivery catheter is utilized to deploy anchoring devices that are configured to support a prosthetic heart valve at an implant site, and methods of using the delivery catheter to implant such anchoring devices and/or prosthetic heart valves.

### BACKGROUND

Blood flow between heart chambers is regulated by native valves—the mitral valve, the aortic valve, the pulmonary valve, and the tricuspid valve. Each of these valves is a passive one-way valve that opens and closes in response to differential pressures. Patients with valvular disease have abnormal anatomy and/or function of at least one valve. For example, a valve may suffer from insufficiency, also referred to as regurgitation, when the valve does not fully close, thereby allowing blood to flow retrograde. Valve stenosis can cause a valve to fail to open properly. Other diseases may also lead to dysfunction of the valves.

The mitral valve, for example, sits between the left atrium and the left ventricle and, when functioning properly, allows blood to flow from the left atrium to the left ventricle while preventing backflow or regurgitation in the reverse direction. Native valve leaflets of a diseased mitral valve, however, do not fully prolapse, causing the patient to experience regurgitation.

While medications may be used to treat diseased native valves, the defective valve often needs to be repaired or replaced at some point during the patient's lifetime. Existing prosthetic valves and surgical repair and/or replacement procedures may have increased risks, limited lifespans, and/or are highly invasive. Some less invasive transcatheter options are available, but most are not ideal. A major limitation of existing transcatheter mitral valve devices, for example, is that the mitral valve devices are too large in diameter to be delivered transseptally, requiring transapical access instead. Additionally, challenges exist to provide an anchoring or docking system that is not only sufficiently robust to secure a prosthetic valve within the native valve, but also deliverable in a transcatheter approach. Thus, a new valve delivery system or method that overcomes some or all of these deficiencies is desired.

### SUMMARY

Described herein are devices and methods for use in delivering an anchoring device, for example during a mitral valve replacement. In one aspect of the present disclosure, a method of using a delivery system to deliver an anchoring device to a native valve of a patient's heart includes advancing a sheath of the delivery system into a heart chamber, advancing a delivery catheter of the delivery system through the sheath into the heart chamber, generating a first curved portion in a first bending section of the sheath, generating a second curved portion in a second bending section of the sheath, positioning a distal opening of the delivery catheter near the native valve, and delivering the anchoring device through the distal opening to the native valve. In one embodiment, the first flexing section is within the heart chamber. In one embodiment, the second flexing section is within the heart chamber. In one embodiment, at least one of the first and second flexing sections comprises a shapeset configuration. In one embodiment, at least one of generating the first curved portion and generating the second curved portion comprises actuating a pull line. In one embodiment, the first flexing section is generally within a first plane and the second flexing section is generally within a second plane. In one embodiment, the first plane is non-coplanar to the second plane. In one embodiment, the first plane is generally orthogonal to the second plane. In one embodiment, positioning comprises adjusting a curvature of the first curved portion and/or the second curved portion. In one embodiment, positioning comprises advancing and/or retracting the delivery catheter within the sheath. In one embodiment, the method further includes forming an anchor guide at a distal portion of the delivery catheter within the heart chamber. In one embodiment, forming the anchor guide comprises generating at least one anchor guide curve in a portion of the delivery catheter. In one embodiment, generating the at least one anchor guide curve comprises actuating a pull line. In one embodiment, generating the at least one anchor guide curve comprises the anchor guide moving toward a shapeset configuration. In one embodiment, positioning the distal opening further comprises positioning at least a portion of the delivery catheter to be parallel to a plane of an annulus of the native valve. In one embodiment, positioning the distal opening is in a direction of a commissure of the native valve. In one embodiment, positioning the distal opening is below a plane of an annulus of the native valve.

In one aspect of the present disclosure, a delivery system for delivering a prosthesis to a native valve of a patient's heart includes a steerable outer sheath comprising a shaft having a first bending region and a second bending region in a distal end, and an inner shaft disposed within the outer sheath, the inner shaft comprising an anchor guide having a distal end with a distal opening for delivery of an anchor therefrom, wherein the steerable outer sheath is configured such that actuation of at least one of the first and second bending regions forms at least a first curved portion or a second curved portion along the shaft to adjust a position of the inner shaft distal end for directing delivery of the anchor to the native valve. In one embodiment, at least a portion of the first bending region or the second bending region comprises a shapeset configuration at a corresponding first curvature or second curvature. In one embodiment, the steerable outer sheath comprises a pull line coupled to a pull ring adapted to actuate one of the first bending region or the second bending region. In one embodiment, the steerable outer sheath comprises at least two pull lines coupled to at least two respective pull rings that adapted to actuate the first bending region and the second bending region. In one embodiment, the at least two pull lines coupled and the at least two respective pull rings are arranged for independent actuation of the first and second bending regions. In one embodiment, the anchor guide is formed to have a geometry to direct an anchor delivered from the distal tip in a direction generally radially outward from the outer sheath distal end. In one embodiment, the inner shaft is configured to rotate relative to the outer sheath. In one embodiment, at least one of the first bending region and the second bending region comprises a laser cut hypotube.

### BRIEF DESCRIPTION OF THE DRAWINGS

The foregoing and other objects, features, and advantages of the invention will become more apparent from the following detailed description using the accompanying figures. In the drawings:

FIGS. 1A-IG illustrate various stages of delivering an exemplary anchoring device to a position within a native valve using an exemplary delivery system of the present disclosure;

FIG. 2 illustrates an exemplary delivery guide catheter (e.g., sheath) and handle that provides steering control of a distal portion thereof;

FIG. 3 illustrates an anchor guide catheter and handle according to an embodiment of the present disclosure;

FIG. 4 illustrates a device for loading an anchor guide catheter into a delivery catheter according to an embodiment of the present disclosure; and

FIG. 5 illustrates an arrangement of an anchor guide catheter and a delivery guide catheter according to an embodiment of the present disclosure.

### DETAILED DESCRIPTION

The following description and accompanying figures, which describe and show certain embodiments, are made to demonstrate, in a non-limiting manner, several possible configurations of systems, devices, apparatuses, components, methods, etc. that may be used for various aspects and features of the present disclosure. As one example, various systems, devices/apparatuses, components, methods, etc. are described herein that may relate to mitral valve procedures. However, specific examples provided are not intended to be limiting, e.g., the systems, devices/apparatuses, components, methods, etc. can be adapted for use in other valves beyond the mitral valve (e.g., in the tricuspid valve).

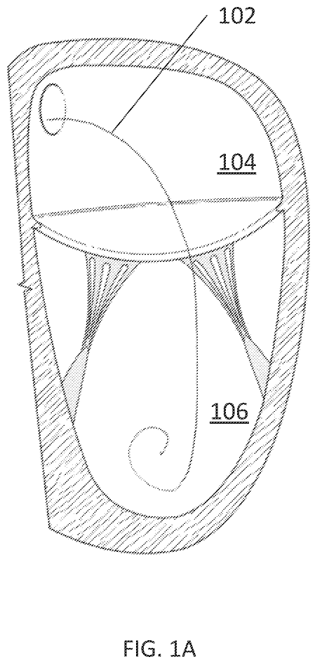

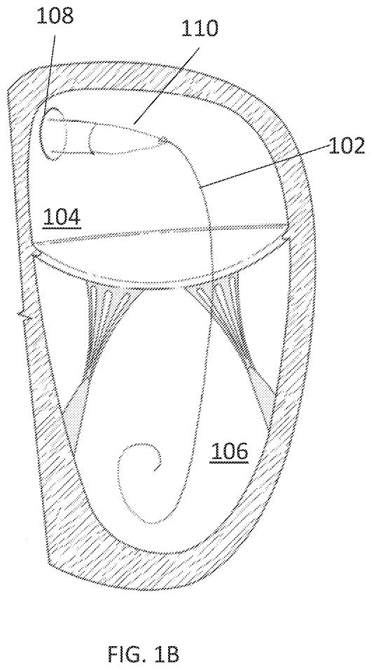

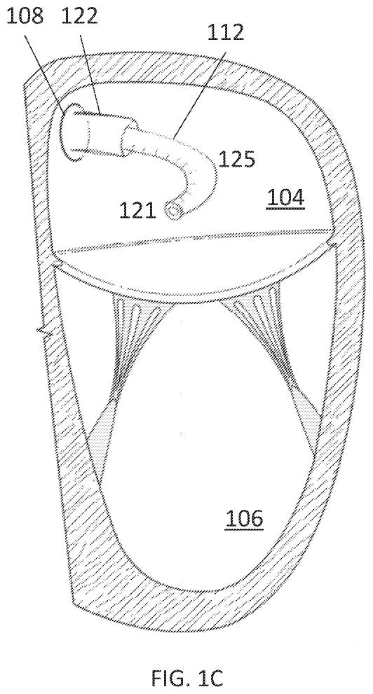

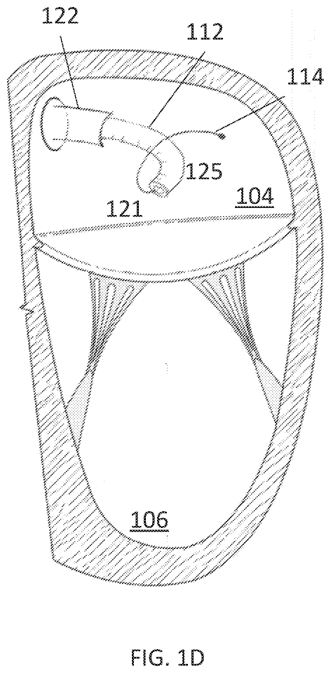

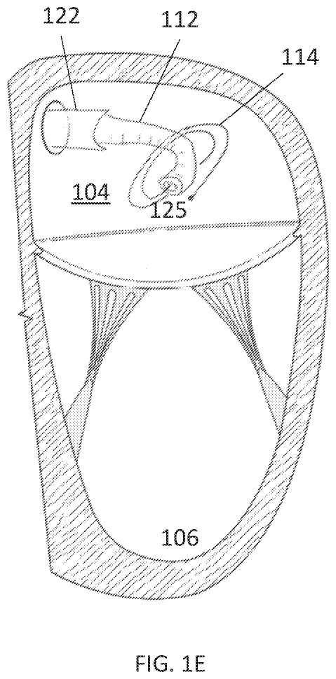

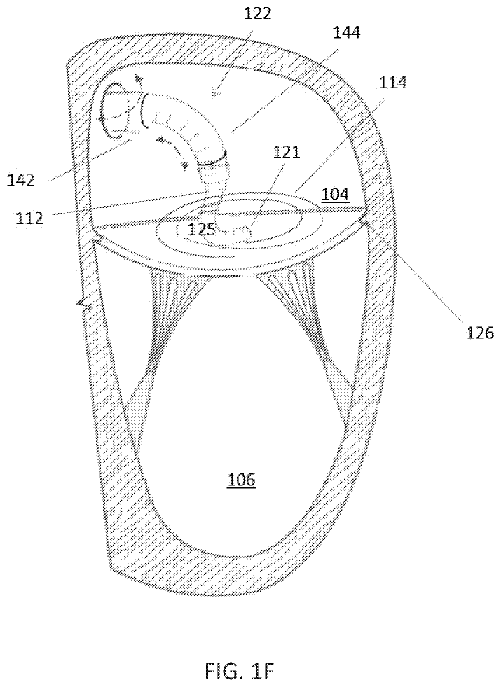

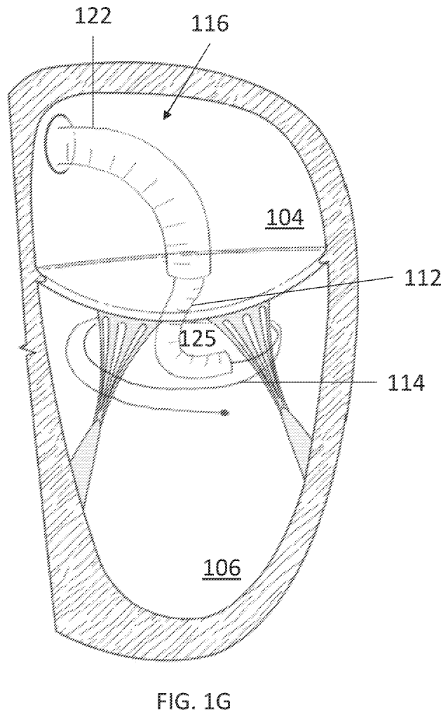

FIGS. 1A-IG show an exemplary method of delivering an anchor of a valve system. At FIG. 1A, a transseptal puncture is made. A guidewire 102 is then routed through the puncture site and left either in the left atrium 104 or across the mitral valve into the left ventricle 106. At FIG. 1B, an outer sheath 108 (optionally with an inner dilator 110) is tracked over the guidewire 102 until the distal end of the outer sheath 108 protrudes into the left atrium 104. The guidewire 102 and inner dilator 110 are then removed from the outer sheath 108. At FIG. 1C, a steerable guide sheath (e.g., catheter) 122 carrying a guide catheter 112 is inserted through the outer sheath 108 until the distal tip 121 of an anchor guide catheter (e.g., anchor guide 125) extends into the left atrium 104. At FIG. 1D, once the anchor guide 125 is in the correct orientation, the anchor 114 can be pushed out through distal tip 121 of the anchor guide 125. At FIG. 1E, the curvature of the anchor guide 125 can cause torsion on the anchor 114, causing the anchor 114 to deploy concentrically with the outer sheath 108 into the atrium 104. At FIG. 1F the anchor guide 125 and/or anchor 114 can be positioned and/or oriented as desired by a) steering a first bending region 142 and/or a second bending region 144 of the steerable guide sheath 122, b) advancing/retracting the shaft of the guide catheter 112 within the steerable guide sheath 122, c) rotating the shaft of the guide catheter 112 within the steerable guide sheath 122, d) steering the distal tip 121, or e) any combination of the foregoing. At FIG. 1G, the entire delivery system 116 can be pushed and steered (via steering mechanisms in the steerable guide sheath 122 and or the delivery guide catheter 112) towards an apex of the ventricle 106, crossing through the mitral valve. In some embodiments, counter-rotation of the anchor 114 (via counter-rotation of the delivery guide catheter (e.g., inner shaft) 112 and delivery guide 125) may aid in getting the anchor across the mitral valve without entanglement of the adjacent anatomy. Once the anchor 114 is at the correct depth within the ventricle 106, forward rotation of the anchor 114 (via forward rotation of the delivery catheter 112 and delivery guide 125) promotes the anchor 114 to encircle the mitral leaflets and chordae. In some embodiments, the anchor 114 can be deployed towards the apex or papillary muscles to avoid interference with mitral leaflet motion.

As shown in FIGS. 1A-IG, the anchor 114 can be advanced from the delivery catheter 112 within the atrium 104, passed through into the ventricle 106, and deployed via rotation to encircle sub-valvular anatomy. It will be appreciated that the delivery system of the present disclosure can also be adapted such that the anchor 114 is deployed to encircle sub-valvular anatomy as it is advanced (e.g., extruded) from delivery catheter 122, either alone or in combination with rotation thereof. For example, the steerable guide sheath 122 and delivery catheter 112 can be advanced to place at least a portion of the delivery guide 125 in the sub-annular space prior to advancement of anchor 114 from the distal tip 121. Once the distal tip 121 is confirmed to be positioned in a selected manner, anchor 114 can be advanced from the delivery catheter 112 within the ventricle 106 to encircle sub-valvular anatomy. In some embodiments, the steerable guide sheath 122 and delivery catheter 112 are manipulated to orient the distal tip 121 within the atrium and toward a commissure 126, and the anchor 114 is delivered through the commissure 126 (e.g., A3P3) of the heart chamber to encircle sub-valvular anatomy.

Embodiments of methods and devices for delivering an anchor/valve prosthesis are described in U.S. patent application Ser. No. 16/824,596, filed Mar. 19, 2020, and U.S. patent application Ser. No. 15/594,946, filed Oct. 7, 2019, the entire disclosures of which are incorporated by reference herein.

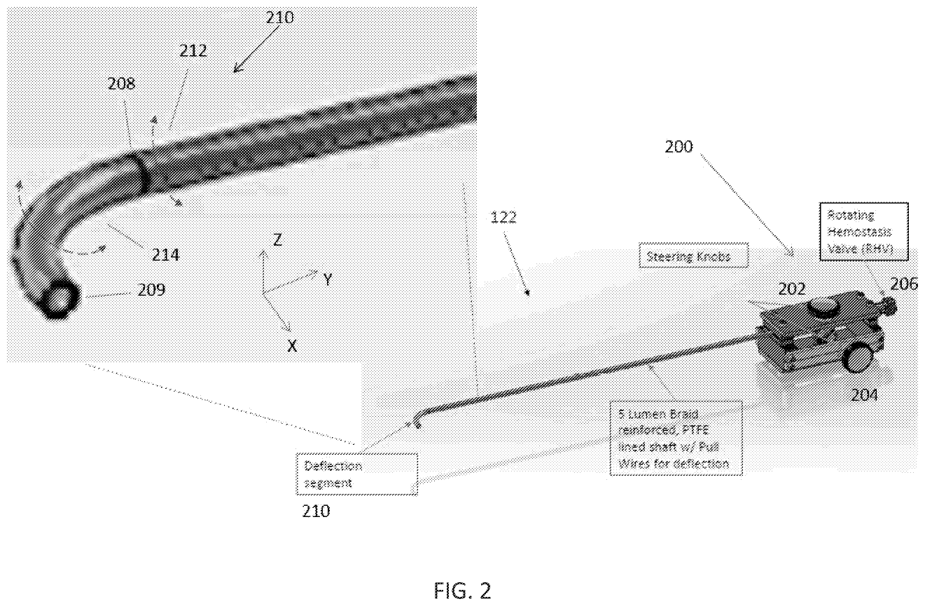

Referring to FIG. 2, a steerable guide sheath 122 can be used to steer the anchor and/or delivery components such that they are positioned at a proper location and orientation with respect to the native heart. For example, the steerable guide sheath 122 can position the delivery catheter 112 and/or delivery guide 125 to be generally central to the mitral valve annulus. The steerable guide sheath 122 can, for example, include a shaft having one or more pull wires and pull rings carried therein configured to deflect a distal portion of the guide sheath 122. As shown in FIG. 2, a distal end 210 of the steerable guide sheath 122 includes a first pull ring 208 and a second pull ring 209 (connecting pull lines not shown). Pull lines are connected to the pull rings and routed proximally to controls (e.g., 202 and 204) of a handle 200 of the steerable guide sheath 122. The handle 200 can include a (e.g., rotatable) hemostasis valve 206 that enables introduction of delivery devices, for example an inner catheter (e.g., delivery catheter 112) and anchor 114. Deflection of distal end 210 can be controlled by manipulation of one or both of controls 202 and 204, which can each have 2-way actuation (e.g., to enable 4-way steering). As shown in FIG. 2, actuation of pull ring 208 generates deflection in a y-z plane in a first bending section 212, while actuation of pull ring 209 generates deflection in a x-y plane in a bending section 214. While shown as being orthogonal in FIG. 2, it will be appreciated that bending sections 212 and 214 can be oriented in planes that are non-orthogonal.

In some embodiments, a deflection angle of the first bending section 212 and/or the second bending section 214 can be about 30 degrees. In some embodiments, a deflection angle of the steerable sheath 122 can be moved between about 0 degrees and about 90 degrees, such as, for example, between about 5 degrees and about 80 degrees, such as between about 10 degrees and 70 degrees, such as between about 15 degrees and about 60 degrees, such as between about 20 degrees and about 50 degrees, such as between about 25 degrees and about 40 degrees, such as between about 27 degrees and about 33 degrees.

The distal section 210 can have two or more actuation points, each of which can be actuated independently. An exemplary actuation mechanism includes separate pull wires that are controlled by separate controls (e.g., knobs, tabs, inputs, buttons, levers, switches, etc.) or other mechanisms. The first and second bending sections are configured to generate deflections (e.g., curvature) in the steerable guide sheath 122 that place the delivery catheter 112 and delivery guide 125 in prescribed locations for delivery of the anchor 114. For example, the steerable guide sheath 122 can be deflected to place the distal end 121 of the delivery guide 125 to be pointed towards the mitral plane, and the (generally planar) curved portion of the delivery guide 125 to curve generally around the mitral annulus.

In some embodiments, the distal end 210 of the guide sheath 122 can be formed to produce differential deflection. Deflection can include non-constant curvature in a given bending section, and/or compound (e.g., non-planar) curvature. In some embodiments, at least one bending section of the distal section 210 is formed from a laser cut hypotube. In some embodiments, at least one bending section of the distal section 210 is formed comprising a polyether block amide (PEBAX) that is coated over a coiled or braided tube. In some embodiments, the first bending section can extend to the distal tip of the delivery catheter and be constructed, for example, with PEBAX having a hardness of approximately 55D that is reflowed over a coiled or braided tube. The second flexible section can also be constructed, for example, with PEBAX, with for example a hardness of approximately 50D, and that is also reflowed over a coiled or braided tube. Using this configuration can provide a distal section 210 that can be shaped and actuated substantially similarly to that of a laser cut hypotube, without the need to form the entire distal section 210 as a laser cut hypotube, or any portion from a laser cut hypo tube.

Steering of the distal end 210 of the steerable sheath 122 can advantageously adjust a position and/or orientation of a delivery catheter 112, for example to place a delivery guide 125 into a proper alignment to delivery an anchor 114 to encircle tissue of the native valve, while obviating the need to steer/tension the delivery catheter 112 itself. Steering the delivery catheter 112 during delivery of the anchor 114 can increase the forces required to deliver the anchor 114, as the anchor 114 must advance through a tensioned device. In certain embodiments, steering the delivery catheter 112 during delivery of the anchor 114 can deform the anchor 114 from its intended (e.g., shapeset) configuration, increasing a risk of undesirable tissue entanglement or preventing successful anchor encircling of native tissue. Embodiments of a delivery system of the present disclosure provide a steerable sheath 122 that can be actuated to adjust an angle of the anchor 114 with respect to the mitral annulus (e.g., adjust to point down/parallel with plane of mitral annulus).

In some embodiments, at least two pull rings can be connected by a (e.g., embedded) spine that is implemented on a radially opposite side of one of the pull wires, for example, opposite a pull wire for the pull ring 209. Such an embedded spine can restrict the relative movement between the pull rings 208 and 209, and better control the direction of deflection caused by pulling the pull wire for the distalmost pull ring 209. Further, an embedded spine can reduce unwanted deflection of the steerable guide sheath 122 that leads to movement of the delivery guide 125 in a direction perpendicular to the mitral plane, or in otherwise unintended directions. While the embodiment described above can include two pull rings and two pull wires, it should be understood that any number of pull rings and/or pull wires can be used to create the various configurations described herein. In addition, it will be appreciated that any suitable number of spines can be used to restrict the relative movement between the pull rings.

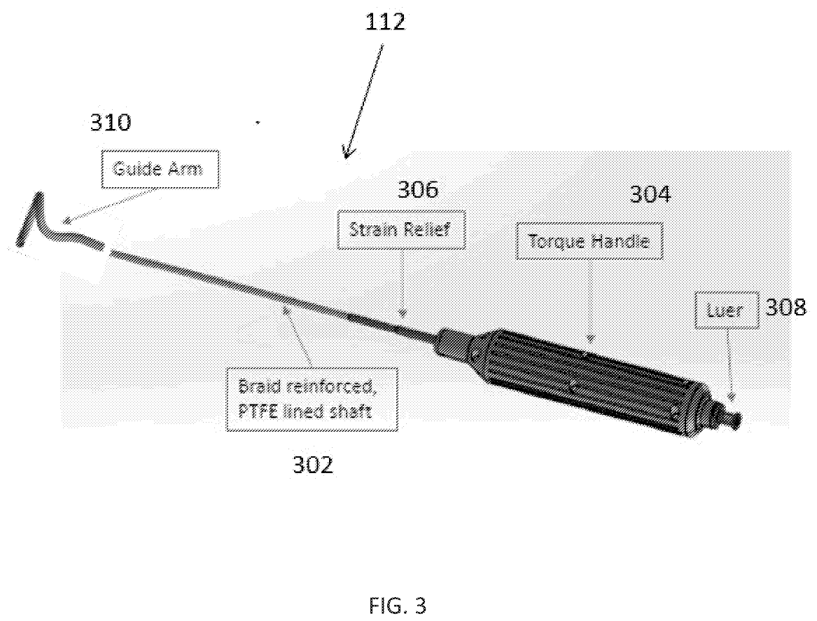

Referring to FIG. 3, an exemplary anchor guide catheter 112 is depicted that is shaped and sized to pass through a steerable guide sheath (e.g., 122). The anchor guide catheter 112 can include a guide arm 310 at the distal end, the guide arm 310 having a three-dimensional geometry that is configured to engage and control the shape and placement of an anchor during deployment in the native heart. For example, the guide arm 310 can have a geometry that directs an anchor in a direction generally radially outward from the shaft of the delivery catheter and/or steerable guide sheath. The exemplary anchor guide catheter 112 of FIG. 3 includes an elongate shaft 302 having a lubricious liner, a proximal portion 306 of the shaft that incorporates strain relief to control undesirable whipping and/or contortion during use, a handle 304 adapted to manipulate the shaft 302 and guide arm 310, and a connection 308 that provides a hemostatic port through which an anchor can be manipulated.

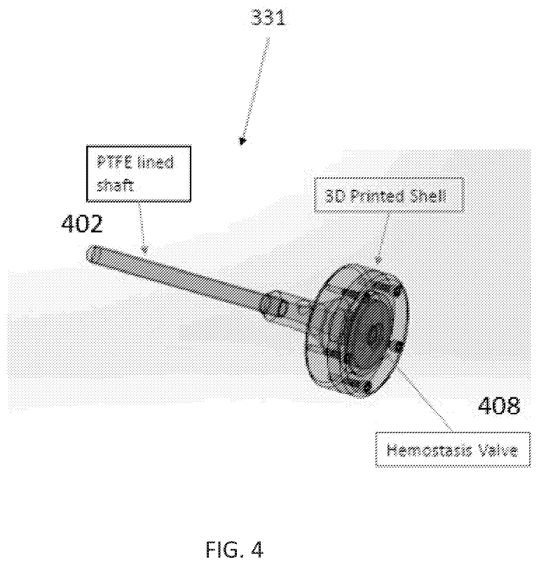

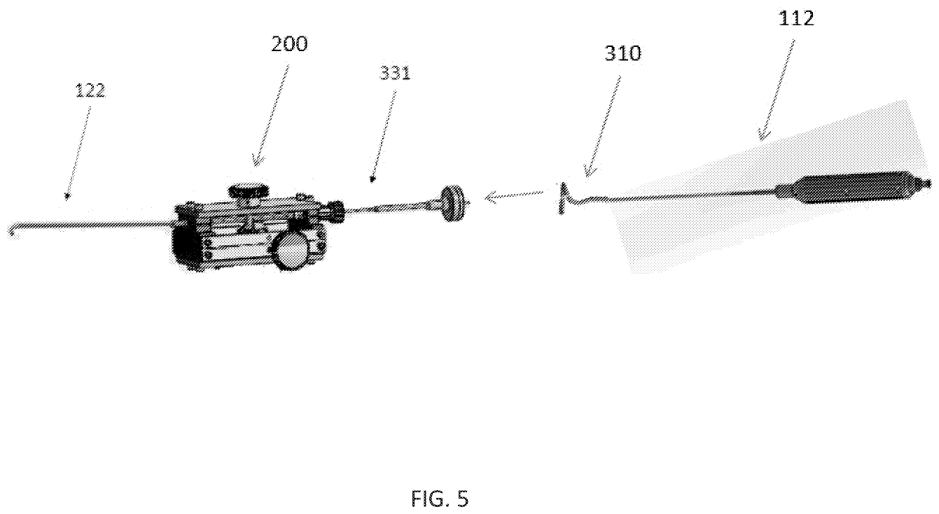

Referring to FIG. 4, a loading device 331 that can be used in order to load the three-dimensional guide arm (e.g., 310) of the anchor guide catheter 112 into the steerable guide catheter 122 is depicted. As depicted in FIG. 5, the loading device 331 can be configured to straighten the guide arm 310 such that it can be inserted through the rotating hemostasis valve at the proximal end of the handle 200 of the steerable guide catheter 122. In some embodiments, the loading device 331 can alternatively or additionally be used to load additional components. For example, the loading device 331 can be used to load the anchor 114 into the distal end of the delivery guide 310.

It should be understood that any feature described herein with respect to one embodiment can be used in addition to or in place of any feature described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected,” “attached” or “coupled” to another feature or element, it can be directly connected, attached or coupled to the other feature or element or intervening features or elements may be present. In contrast, when a feature or element is referred to as being “directly connected,” “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “I”.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise,” and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments many be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of using a delivery system to deliver an anchoring device to a native valve of a patient's heart, the method comprising:
advancing a sheath of the delivery system into a heart chamber;
advancing a delivery catheter of the delivery system through the sheath into the heart chamber;
generating a first curved portion in a first bending section of the sheath;
generating a second curved portion in a second bending section of the sheath;
positioning a distal opening of the delivery catheter near the native valve; and
delivering the anchoring device through the distal opening to the native valve.

2. The method of claim 1, wherein the first flexing section is within the heart chamber.

3. The method of claim 1, wherein the second flexing section is within the heart chamber.

4. The method of claim 1, wherein at least one of the first and second flexing sections comprises a shapeset configuration.

5. The method of claim 1, wherein at least one of generating the first curved portion and generating the second curved portion comprises actuating a pull line.

6. The method of claim 1, wherein the first flexing section is generally within a first plane and the second flexing section is generally within a second plane.

7. The method of claim 6, wherein the first plane is non-coplanar to the second plane.

8. The method of claim 7, wherein the first plane is generally orthogonal to the second plane.

9. The method of claim 1, wherein positioning comprises adjusting a curvature of the first curved portion and/or the second curved portion.

10. The method of claim 1, wherein positioning comprises advancing and/or retracting the delivery catheter within the sheath.

11. The method of claim 1, further comprising forming an anchor guide at a distal portion of the delivery catheter within the heart chamber.

12. The method of claim 11, wherein forming the anchor guide comprises generating at least one anchor guide curve in a portion of the delivery catheter.

13. The method of claim 12, wherein generating the at least one anchor guide curve comprises actuating a pull line.

14. The method of claim 12, wherein generating the at least one anchor guide curve comprises the anchor guide moving toward a shapeset configuration.

15. The method of claim 1, wherein positioning the distal opening further comprises positioning at least a portion of the delivery catheter to be parallel to a plane of an annulus of the native valve.

16. The method of claim 1, wherein positioning the distal opening is in a direction of a commissure of the native valve.

17. The method of claim 1, wherein positioning the distal opening is below a plane of an annulus of the native valve.

18. A delivery system for delivering a prosthesis to a native valve of a patient's heart, the delivery system comprising:
a steerable outer sheath comprising a shaft having a first bending region and a second bending region in a distal end; and
an inner shaft disposed within the outer sheath, the inner shaft comprising an anchor guide having a distal end with a distal opening for delivery of an anchor therefrom,
wherein the steerable outer sheath is configured such that actuation of at least one of the first and second bending regions forms at least a first curved portion or a second curved portion along the shaft to adjust a position of the inner shaft distal end for directing delivery of the anchor to the native valve.

19. A system according to claim 18, wherein at least a portion of the first bending region or the second bending region comprises a shapeset configuration at a corresponding first curvature or second curvature.

20. A system according to claim 19, wherein the first curvature is generally orthogonal to the second curvature.

21. A system according to claim 19, wherein at least one of the first curvature and the second curvature is a compound curve.

22. A system according to claim 18, wherein the steerable outer sheath comprises a pull line coupled to a pull ring adapted to actuate one of the first bending region or the second bending region.

23. A system according to claim 18, wherein the steerable outer sheath comprises at least two pull lines coupled to at least two respective pull rings that adapted to actuate the first bending region and the second bending region.

24. A system according to claim 23, wherein the at least two pull lines coupled and the at least two respective pull rings are arranged for independent actuation of the first and second bending regions.

25. A system according to claim 18, wherein the anchor guide is formed to have a geometry to direct an anchor delivered from the distal tip in a direction generally radially outward from the outer sheath distal end.

26. A system according to claim 18, wherein the inner shaft is configured to rotate relative to the outer sheath.

27. A system according to claim 18, wherein at least one of the first bending region and the second bending region comprises a laser cut hypotube.
